(19) United States
(12) Patent Application Publication
(10) Pub. No.: US 2008/0058417 A1
(43) Pub. Date: Mar. 6, 2008

(54) Enriched Beverages and Methods of Making The Same

(75) Inventors: Jesus Ruben Abril, Westminster, CO (US); Thayne Fort, Denver, CO (US)

Correspondence Address: SHERIDAN ROSS P.C., 1560 BROADWAY, SUITE 1200, DENVER, CO, 80202, US

(73) Assignee: MARTEK BIOSCIENCES CORPORATION, Columbia, CO (US)

(21) Appl. No.: 11/838,316

(22) Filed: Aug. 14, 2007

Related U.S. Application Data
(60) Provisional application No. 60/822,363, filed on Aug. 14, 2006.

(51) Int. Cl. A61K 31/20; A23L 2/02; A61P 9/10

(52) U.S. Cl. 514/558; 426/545; 426/577; 426/590; 426/599

(57) ABSTRACT

The present invention provides a composition that contains a beverage component, pectin and an oil at least one long chain polyunsaturated fatty acid and a method for making the same.

### CROSS-REFERENCE TO RELATED APPLICATION

This application claims the benefit of priority under 35 U.S.C. § 119(e) of U.S. Provisional Application Ser. No. 60/822,363, filed Aug. 14, 2006. The disclosure of this application is incorporated by reference herein in its entirety.

### FIELD OF THE INVENTION

The present invention relates to a beverage stabilizing system and method employing a hydrocolloid, such as pectin, in the production of beverages which are enriched in polyunsaturated fatty acids. The invention also relates to the beverages produced therefrom.

### BACKGROUND OF THE INVENTION

It is desirable to increase the dietary intake of the beneficial polyunsaturated fatty acids, including omega-3 polyunsaturated fatty acids (PUFA), and omega-3 long chain polyunsaturated fatty acids (omega-3 LC PUFAs). Other beneficial nutrients are omega-6 long chain polyunsaturated fatty acids (omega-6 LC PUFA). As used herein, reference to a long chain polyunsaturated fatty acid or LC PUFA, refers to a polyunsaturated fatty acid having 18 or more carbons. Omega-3 PUFAs are recognized as important dietary compounds for preventing arteriosclerosis and coronary heart disease, for alleviating inflammatory conditions, cognitive impairment and dementia-related diseases and for retarding the growth of tumor cells. One important class of omega-3 PUFAs is omega-3 LC PUFAs. Omega-6 LC-PUFAs serve not only as structural lipids in the human body, but also as precursors for a number of factors in inflammation such as prostaglandins, and leukotrienes.

Fatty acids are carboxylic acids and are classified based on the length and saturation characteristics of the carbon chain. Short chain fatty acids have 2 to about 6 carbons and are typically saturated. Medium chain fatty acids have from about 6 to about 18 carbons and may be saturated or unsaturated. Long chain fatty acids have from 18 to 24 or more carbons and may also be saturated or unsaturated. In longer fatty acids there may be one or more points of unsaturation, giving rise to the terms “monounsaturated” and “polyunsaturated,” respectively. LC PUFAs are of particular interest in the present invention.

LC PUFAs are categorized according to the number and position of double bonds in the fatty acids according to a well understood nomenclature. There are two common series or families of LC PUFAs, depending on the position of the double bond closest to the methyl end of the fatty acid: the α-3 (or n-3 or omega-3) series contains a double bond at the third carbon, while the α-6 (or n-6 or omega-6) series has no double bond until the sixth carbon. Thus, docosahexaenoic acid (“DHA”) has a chain length of 22 carbons with 6 double bonds beginning with the third carbon from the methyl end and is designated “22:6 n-3”. Other important LC PUFAs include eicosapentaenoic acid (“EPA”) which is designated “20:5 n-3” and docosapentaenoic acid (“DPAn-3”) which is designated “22:5 n-3,” arachidonic acid (“ARA”) which is designated “20:4 n-6” and docosapentaenoic acid (“DPAn-6”) which is designated “22:5 n-6” are suitable. Other, less common series or families of LC PUFAs exist, such as ω-9 (or n-9 or omega-9) series which has no double bond until the ninth carbon.

De novo or “new” synthesis of the omega-3 and omega-6 fatty acids such as DHA and ARA does not occur in the human body; however, the body can convert shorter chain fatty acids to LC PUFAs such as DHA and ARA, although at very low efficiency. Omega-3 and omega-6 fatty acids must be part of the nutritional intake since the human body cannot insert double bonds closer to the omega end than the seventh carbon atom counting from that end of the molecule. Thus, all metabolic conversions occur without altering the omega end of the molecule that contains the omega-3 and omega-6 double bonds. Consequently, omega-3 and omega-6 acids are two separate families of essential fatty acids that are not interconvertible in the human body.

Over the past few decades, health experts have recommended diets lower in saturated fats and higher in polyunsaturated fats. While this advice has been followed by a number of consumers, the incidence of heart disease, cancer, diabetes and many other debilitating diseases has continued to increase steadily. Scientists agree that the type and source of polyunsaturated fats is as critical as the total quantity of fats. The most common polyunsaturated fats are derived from vegetable matter and are lacking in long chain fatty acids (most particularly omega-3 LC-PUFAs). In addition, the hydrogenation of polyunsaturated fats to create synthetic fats has contributed to the rise of certain health disorders and exacerbated the deficiency in some essential fatty acids. Indeed, many medical conditions have been identified as benefiting from an omega-3 supplementation. These include acne, allergies, Alzheimer's, arthritis, atherosclerosis, breast cysts, cancer, cystic fibrosis, diabetes, eczema, hypertension, hyperactivity, intestinal disorders, kidney dysfunction, leukemia, and multiple sclerosis. Of note, the World Health Organization has recommended that infant formulas be enriched with omega-3, and omega-6, fatty acids.

The polyunsaturates derived from meat contain significant amounts of omega-6 but little or no omega-3. While omega-6 and omega-3 fatty acids are both necessary for good health, they are preferably consumed in a balance of about 4:1. Today's Western diet has created a serious imbalance with current consumption on average of 20 times more omega-6 than omega-3. Concerned consumers have begun to look for health food supplements to restore the equilibrium. Principal sources of omega-3 are flaxseed oil and fish oils. The past decade has seen rapid growth in the production of flaxseed and fish oils. Both types of oil are considered good dietary sources of omega-3 polyunsaturated fats. Flaxseed oil contains no EPA, DHA, or DPA but rather contains linolenic acid—a building block that can be elongated by the body to build longer chain PUFAs. There is evidence, however, that the rate of metabolic conversion can be slow and unsteady, particularly among those with impaired health. Fish oils vary considerably in the type and level of fatty acid composition depending on the particular species and their diets. For example, fish raised by aquaculture tend to have a lower level of omega-3 fatty acids than fish from the wild. In light of the health benefits of omega-3 LC PUFAs and other LC PUFAs, it would be desirable to supplement foods with such fatty acids.

Due to the scarcity of sources of omega-3 LC PUFAs, typical home-prepared and convenience foods are low in both omega-3 PUFAs and omega-3 LC PUFAs (carbon chain length greater than 18, and preferably greater than 20), such as docosahexaenoic acid, docosapentaenoic acid, and eicosapentaenoic acid. In light of the health benefits of such omega-3 LC PUFAs and of other LC PUFAs, it would be desirable to supplement foods with such fatty acids.

In light of the desirability of supplementing foods with LC PUFAs and in view of the shortcomings of the prior art in providing these foods, there is a need for methods for enriching foods with LC PUFAs and also for food oil compositions and food products comprising LC PUFAs. These and other needs are answered by the present invention.

### SUMMARY OF THE INVENTION

The present invention is directed toward compositions, including beverages, having a high content of LC PUFAs and methods of producing the same. More particularly, the present invention includes a composition comprising a hydrocolloid, such as pectin, an oil comprising at least one long chain polyunsaturated fatty acid, and a beverage component. The compositions of the invention are stable preparations, which lack alginate and calcium gluconate.

In preferred embodiments, long chain polyunsaturated fatty acid is selected from the group consisting of omega-3 and omega-6 LC PUFAs. The omega-3 LC PUFA or omega-6 LC PUFA can be docosahexaenoic acid, eicosapentaenoic acid, docosapentaenoic acid or arachidonic acid and mixtures thereof. The omega-3 LC PUFA or omega-6 containing oil preferably can be from a microbial source, such as a microorganism selected from algae, protists, bacteria or fungi and/or an oleaginous microorganism. The microbial source preferably can be a microorganism selected from microorganisms of the genus Thraustochytrium, microorganisms of the genus Schizochytrium, microorganisms of the genus Althomia, microorganisms of the genus Aplanochytrium, microorganisms of the genus Japonochytrium, microorganisms of the genus Elina, microorganisms of the genus Crypthecodinium, and in preferred embodiments is a microorganism from microorganisms of the genus Schizochytrium, and microorganisms of the genus Mortierella, and mixtures thereof.

Alternatively, the omega-3 LC PUFA or omega-6 LC PUFA containing oil can be from a plant source, such as a from soybean, corn, safflower, sunflower, canola, flax, peanut, mustard, rapeseed, chickpea, cotton, lentil, white clover, olive, palm, borage, evening primrose, linseed and tobacco. The plants can be either genetically modified to produce long chain polyunsaturated fatty acids or not.

The oil can alternatively be from an animal source, such as aquatic animals, animal tissues or animal products. The oil preferably can include at least about 20%, at least about 30%, at least about 40%, at least about 50%, at least about 60%, at least about 70%, or at least about 80% omega-3 LC PUFAs or omega-6 LC PUFAs.

In an embodiment of the invention, the omega-3 LC PUFA containing oil can be minimally processed. In a further embodiment, the oil not winterized. In a preferred embodiment of the invention, the oil can be encapsulated in a powder.

A composition of the invention preferably can include between about 5 mg and about 1000 mg; between about 5 mg and about 250 mg of omega-3 LC PUFA or omega-6 LC PUFA per serving and between about 5 mg and about 100 mg of omega-3 LC PUFA or omega-6 LC PUFA per serving. More preferably, the compositions; between about 50 mg and about 150 mg of omega-3 LC PUFA or omega-6 LC PUFA per serving and between about 75 mg and about 125 mg of omega-3 LC PUFA or omega-6 LC PUFA per serving.

A composition of the invention can also include an antioxidant, which preferably can be vitamin E, butylhydroxytoluene (BHT), butylhydroxyanisole (BHA), tertbutylhydroquinone (TBHQ), propyl gallate (PG), vitamin C, a phospholipid, or a natural antioxidant, and in a preferred embodiment is TBHQ. The antioxidant preferably can be present in an amount of between about 0.01% and about 0.2% by weight of the oil or between about 0.05% and about 0.15% by weight of the oil.

Pectins useful in the invention include, but are not limited to, natural pectins and modified pectin. Preferably, the pectin is pre-hydrated pectin. In various embodiments of the invention, the pectin can be present in an amount of about 0.05% to 0.20% by weight of the composition, about 0.03 to 0.3% by weight of the composition, or about 0.1% to 0.5% by weight of the composition.

The compositions of the invention also include a beverage component. The beverage component can comprise a natural flavor or an artificial flavor or a mixture thereof. In preferred embodiments, the beverage component is selected from the group consisting of fruit juice, fruit flavor, fruit concentrate, tea, water, carbonated water, protein and mixtures thereof. In a more preferred embodiment, the beverage component is a fruit juice. In a still more preferred embodiment, the fruit juice is orange juice. The fruit juice can be concentrated or unconcentrated.

Additional embodiments of the invention include methods for preparing the compositions of the invention, and the compositions prepared by the methods. A preferred method of the present invention comprises combining an oil comprising an omega-3 LC PUFA and/or omega-6 LC PUFA with pectin and a beverage component.

In a preferred embodiment, the method comprises combining an omega-3 LC PUFA, an omega-6 LC PUFA, or an omega-3/omega-6 LC PUFA-containing oil with a hydrocolloid compound, such as pectin, to produce a pre-emulsion, which will be an oil in water emulsion. The pre-emulsion can then be added to a beverage component to generate a beverage that is enriched with at least one omega-3 or omega-6 LC PUFA or a mixture thereof. In this manner, the omega-3 LC PUFA and/or omega-6 LC PUFA is stabilized during the storage phase of the beverage.

In another embodiment of the invention, which is particularly useful in the preparation of a juice, such as a 100% juice, pectin can be added to the juice, preferably under high shear, and the oil comprising at least one omega-3 LC PUFA and/or omega 6-LC PUFA is added. The entire mixture (the pectin/oil/juice) can then be homogenized.

In yet another embodiment of the invention, pectin can be combined, under high shear, with water and an omega-3 LC PUFA containing oil added thereto, under high shear. This mixture can then be added to a desired amount of a beverage, and the mixture generated therefrom can then be subjected to homogenization. In this embodiment, the whole fortified/enriched beverage is subjected to homogenization.

### DETAILED DESCRIPTION OF THE INVENTION

The compositions, products and methods of the present invention provide beverages that have an enhanced nutritional value. The beverages, which have enriched LC PUFA content (including for example enriched omega-3 and/or omega-6 LC PUFA content) provide for increased intake of LC PUFAs by those consuming them. The beverages can therefore provide health benefits. The present invention also provides methods to minimize the oxidative degradation of LC PUFAs, including omega-3 LC PUFAs and omega-6 LC PUFAs, in the compositions and products of the invention.

In various embodiments, the present invention is directed toward compositions that have high contents of omega-3 LC PUFAs and/or omega-6 LC PUFAs and methods of producing the same.

In one embodiment, the invention provides a composition which is comprised of an oil comprising at least one omega-3 or omega-6 long chain fatty acid, or a mixture thereof, a pectin, and a beverage component. The composition is preferably free of alginate and of calcium gluconate. The composition is preferably a beverage. As used herein, the term “comprising” means various components can be conjointly employed in the composition. Accordingly, the terms “comprising,” “comprised of,” “comprise,” and “comprises” encompass the more restrictive terms “consisting essentially of” and “consisting of.”

The oils of the invention preferably comprise at least one omega-3 LC PUFA, at least one omega-6 LC PUFA or mixtures thereof. Preferred omega-3 LC PUFAs include, for example, docosahexaenoic acid C22:6(n-3) (DHA), eicosapentaenoic acid C20:5(n-3) (EPA), and omega-3 docosapentaenoic acid C22:5(n-3) (DPAn-3). DHA is particularly preferred. Preferred omega-6 LC-PUFAs include arachidonic acid C20:4(n-6) (ARA) and docosapentaenoic acid C22:5(n-6) (DPAn-6). The PUFAs can be in any of the common forms found in natural lipids including but not limited to triacylglycerols, diacylglycerols, monoacylglycerols, phospholipids, free fatty acids, esterified fatty acids, or in natural or synthetic derivative forms of these fatty acids (e.g. calcium salts of fatty acids, ethyl esters, etc).

As noted above, LC PUFAs include PUFAs having 18 or more carbons and in preferred embodiments, the omega-3 LC PUFA and omega-6 LC PUFA have 20 or more carbons or 22 or more carbons. Reference to an oil comprising an omega-3 or omega-6 LC PUFA, as used in the present invention, can refer to either an oil comprising only a single omega-3 or omega-6 LC PUFA, such as DHA or ARA or an oil comprising a mixture of omega-3 or omega-6 LC PUFAs, such as a mixture of DHA and EPA or a mixture of ARA and DPAn-6. Fatty acids other than omega-3 and omega-6 fatty acids are also typically present in oils.

A preferred source of oils that comprise omega-3 LC PUFAs, omega-6 LC PUFAs and/or omega-3/omega-6 LC PUFAs in the compositions and methods of the present invention includes a microbial source. PUFAs produced by microorganisms can be used in the methods and compositions of the present invention. In some embodiments, organisms include those selected from the group consisting of golden algae (such as microorganisms of the kingdom Stramenopiles), green algae, diatoms, dinoflagellates (such as microorganisms of the order Dinophyceae including members of the genus Crypthecodinium such as, for example, Crypthecodinium cohnii), yeast, and fungi of the genera Mucor and Mortierella, including but not limited to Mortierella alpina and Mortierella sect. schmuckeri. Members of the microbial group Stramenopiles include microalgae and algae-like microorganisms, including the following groups of microorganisms: Hamatores, Proteromonads, Opalines, Develpayella, Diplophrys, Labrinthulids, Thraustochytrids, Biosecids, Oomycetes, Hypochytridiomycetes, Commation, Reticulosphaera, Pelagomonas, Pelagococcus, Ollicola, Aureococcus, Parmales, Diatoms, Xanthophytes, Phaeophytes (brown algae), Eustigmatophytes, Raphidophytes, Synurids, Axodines (including Rhizochromulinaales, Pedinellales, Dictyochales), Chrysomeridales, Sarcinochrysidales, Hydrurales, Hibberdiales, and Chromulinales. The Thraustochytrids include the genera Schizochytrium (species include aggregatum, limnaceum, mangrovei, minutum, octosporum), Thraustochytrium (species include arudimentale, aureum, benthicola, globosum, kinnei, motivum, multirudimentale, pachydermum, proliferum, roseum, striatum), Ulkenia (species include amoeboidea, kerguelensis, minuta, profunda, radiate, sailens, sarkariana, schizochytrops, visurgensis, yorkensis), Aplanochytrium (species include haliotidis, kerguelensis, profunda, stocchinoi), Japonochytrium (species include marinum), Althornia (species include crouchii), and Elina (species include marisalba, sinorifica). Since there is some disagreement among experts as to whether Ulkenia is a separate genus from the genus Thraustochytrium, for the purposes of this application, the genus Thraustochytrium will include Ulkenia. The Labrinthulids include the genera Labyrinthula (species include algeriensis, coenocystis, chattonii, macrocystis, macrocystis atlantica, macrocystis macrocystis, marina, minuta, roscoffensis, valkanovii, vitellina, vitellina pacifica, vitellina vitellina, zopfi), Labyrinthomyxa (species include marina), Labyrinthuloides (species include haliotidis, yorkensis), Diplophrys (species include archeri), Pyrrhosorus* (species include marinus), Sorodiploph ys* (species include stercorea), Chlamydomyxa* (species include labyrinthuloides, montana). (*=there is no current general consensus on the exact taxonomic placement of these genera).

Suitable microorganisms include those capable of producing lipids comprising the labile compounds omega-3 and/or omega-6 polyunsaturated fatty acids, and in particular microorganisms that are capable of producing DHA, DPA, EPA or ARA) will be described. More particularly, preferred microorganisms are algae, such as Thraustochytrids of the order Thraustochytriales, including Thraustochytrium (including Ulkenia) and Schizochytrium and including Thraustochytriales which are disclosed in commonly assigned U.S. Pat. Nos. 5,340,594 and 5,340,742, both issued to Barclay, all of which are incorporated herein by reference in their entirety. More preferably, the microorganisms are selected from the group consisting of microorganisms having the identifying characteristics of ATCC number 20888, ATCC number 20889, ATCC number 20890, ATCC number 20891 and ATCC number 20892. Also preferred are strains of Mortierella schmuckeri (e.g., including ATCC 74371) and Mortierella alpine (e.g., including microorganisms having the identifying characteristics of ATCC 42430). Also preferred are strains of Crypthecodinium cohnii, including microorganisms having the identifying characteristics of ATCC Nos. 30021, 30334-30348, 30541-30543, 30555-30557, 30571, 30572, 30772-30775, 30812, 40750, 50050-50060, and 50297-50300. Oleaginous microorganisms are also preferred. As used herein, “oleaginous microorganisms” are defined as microorganisms capable of accumulating greater than 20% of the dry weight of their cells in the form of lipids. Genetically modified microorganisms that produce PUFAs are also suitable for the present invention. These can include naturally PUFA-producing microorganisms that have been genetically modified as well as microorganisms that do not naturally produce PUFAs but that have been genetically modified to do so.

Suitable organisms can be obtained from a number of available sources, including by collection from the natural environment. For example, the American Type Culture Collection currently lists many publicly available strains of microorganisms identified above. As used herein, any organism, or any specific type of organism, includes wild strains, mutants, or recombinant types. Growth conditions in which to culture or grow these organisms are known in the art, and appropriate growth conditions for at least some of these organisms are disclosed in, for example, U.S. Pat. No. 5,130,242, U.S. Pat. No. 5,407,957, U.S. Pat. No. 5,397,591, U.S. Pat. No. 5,492,938, and U.S. Pat. No. 5,711,983, all of which are incorporated herein by reference in their entirety.

A preferred LC PUFA containing oil, such as one from a microbial source, can preferably have less than 7, less than 6, less than 5 or less than 4 PUFAs, in amounts greater than about 2% by weight, greater than about 2.5% by weight, greater than about 3% by weight or greater than about 3.5% by weight of total fatty acids. Preferred microbial oils that are useful in the present invention include those that are disclosed in U.S. Patent Application No. 60/695,996 (entitled “Polyunsaturated Fatty Acid-Containing Oil Product and Uses and Production Thereof,”) filed Jul. 1, 2005; U.S. Patent Application No. 60/738, 304 (entitled the same), filed Nov. 18, 2005; and U.S. patent application Ser. No. 11/428,277, filed Jun. 30, 2006 (entitled the same), all of which are incorporated by reference herein in their entirety. Some of such oils are not subjected to winterization. A preferred microbial oil is known as Martek DHA™-HM and is produced by a process as disclosed in the foregoing patent applications, including a propanol and water extraction process that produces a product with a semi-solid characteristic.

Another preferred source of oils comprising LC PUFAs includes a plant source, such as oilseed plants. Since plants do not naturally produce LC PUFAs of 20 carbons or longer, plants producing LC PUFAs having 20 or more carbons preferably are those genetically modified to express genes that produce such LC PUFAs. Such genes preferably can include genes encoding proteins involved in the classical fatty acid synthase pathways, or genes encoding proteins involved in the PUFA polyketide synthase (PKS) pathway. The genes and proteins involved in the classical fatty acid synthase pathways, and genetically modified organisms, such as plants, transformed with such genes, are described, for example, in Napier and Sayanova, Proceedings of the Nutrition Society (2005), 64:387-393; Robert et al., Functional Plant Biology (2005) 32:473-479; or U.S. Patent Application Publication 2004/0172682. The PUFA PKS pathway, genes and proteins included in this pathway, and genetically modified microorganisms and plants transformed with such genes for the expression and production of PUFAs are described in detail in: U.S. Pat. No. 6,566,583; U.S. Pat. No. 7,247,461, U.S. Pat. No. 7,211,418, and U.S. Pat. No. 7,217,856, each of which is incorporated herein by reference in its entirety.

Preferred oilseed crops include soybean, corn, safflower, sunflower, canola, flax, peanut, mustard, rapeseed, chickpea, cotton, lentil, white clover, olive, palm oil, borage, evening primrose, linseed, and tobacco that have been genetically modified to produce LC PUFAs as described above.

Genetic transformation techniques for microorganisms and plants are well-known in the art. Transformation techniques for microorganisms are well known in the art and are discussed, for example, in Sambrook et al., 1989, Molecular Cloning: A Laboratory Manual, Cold Spring Harbor Labs Press. A general technique for transformation of dinoflagellates, which can be adapted for use with Crypthecodinium cohnii, is described in detail in Lohuis and Miller, The Plant Journal (1998) 13(3): 427-435. A general technique for genetic transformation of Thraustochytrids is described in detail in U.S. Patent Application Publication No. 20030166207, published Sep. 4, 2003. Methods for the genetic engineering of plants are also well known in the art. For instance, numerous methods for plant transformation have been developed, including biological and physical transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88. In addition, vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119. See also, Horsch et al., Science 227:1229 (1985); Kado, C. I., Crit. Rev. Plant. Sci. 10:1 (1991); Moloney et al., Plant Cell Reports 8:238 (1989); U.S. Pat. No. 4,940,838; U.S. Pat. No. 5,464,763; Sanford et al., Part. Sci. Technol. 5:27 (1987); Sanford, J. C., Trends Biotech. 6:299 (1988); Sanford, J. C., Physiol. Plant 79:206 (1990); Klein et al., Biotechnology 10:268 (1992); Zhang et al., Bio/Technology 9:996 (1991); Deshayes et al., EMBO J., 4:2731 (1985); Christou et al., Proc Natl. Acad. Sci. USA 84:3962 (1987); Hain et al., Mol. Gen. Genet. 199:161 (1985); Draper et al., Plant Cell Physiol. 23:451 (1982); Donn et al., In Abstracts of VIIth International Congress on Plant Cell and Tissue Culture IAPTC, A2-38, p. 53 (1990); D'Halluin et al., Plant Cell 4:1495-1505 (1992) and Spencer et al., Plant Mol. Biol. 24:51-61 (1994).

When oilseed plants are the source of LC PUFAs, the seeds preferably can be harvested and processed to remove any impurities, debris or indigestible portions from the harvested seeds. Processing steps vary depending on the type of oilseed and are known in the art. Processing steps preferably can include threshing (such as, for example, when soybean seeds are separated from the pods), dehulling (removing the dry outer covering, or husk, of a fruit, seed, or nut), drying, cleaning, grinding, milling and flaking. After the seeds have been processed to remove any impurities, debris or indigestible materials, they can be added to an aqueous solution preferably water, and then mixed to produce a slurry. Preferably, milling, crushing or flaking is performed prior to mixing with water. A slurry produced in this manner preferably can be treated and processed the same way as described for a microbial fermentation broth. Size reduction, heat treatment, pH adjustment, pasteurization and other known treatments preferably can be used in order to improve quality (nutritional and sensory).

Another preferred source of oils that comprise LC PUFAs includes an animal source. Examples of animal sources include aquatic animals (e.g., fish, marine mammals, and crustaceans such as krill and other euphausids) and animal tissues (e.g., brain, liver, eyes, etc.) and animal products such as eggs or milk. Techniques for recovery of LC PUFA containing oils from such sources are known in the art.

In various embodiments of the invention, the oil may be encapsulated, for example in a powder. In a preferred embodiment, the oil is microencapsulated. Exemplary encapsulation techniques include, for example, U.S. Patent Appln. No. 60/805,590, entitled Encapsulated Labile Compositions and Methods of Making the Same, filed Jun. 22, 2006.

Preferably, the oil comprises at least about 20% of omega-3 LC PUFA, at least about 30% of omega-3 LC PUFA, at least about 40% of omega-3 LC PUFA, at least about 50% of omega-3 LC PUFA, at least about 60% of omega-3 LC PUFA, 70% of omega-3 LC PUFA, and at least about 80% of omega-3 LC PUFA.

Also preferably, the oil comprises at least about 20% of omega-6 LC PUFA, at least about 30% of omega-6 LC PUFA, at least about 40% of omega-6 LC PUFA, at least about 50% of omega-6 LC PUFA, at least about 60% of omega-6 LC PUFA, 70% of omega-6 LC PUFA, and at least about 80% of omega-6 LC PUFA.

Preferably, the compositions and products of the present invention have an LC PUFA content such that an individual serving of the product has an appropriate amount of LC PUFA per serving. Appropriate amounts of LC PUFA per serving are known in the art. Preferred amounts include between about 5 mg per serving and about 1000 mg per serving; between about 5 mg per serving and about 500 mg per serving; between about 5 mg per serving and about 250 mg per serving; and between about 5 mg per serving and about 100 mg per serving. Additional preferred amounts of LC PUFA per serving include amounts of LC PUFA between about 50 mg per serving and about 150 mg per serving; between about 75 mg per serving and about 125 mg per serving. In some medical food applications dosages greater than 1000 mg/serving and greater than 2000 mg/serving may be desirable. Preferred omega-3 LC PUFAs include DHA, EPA and DPAn-3. Preferred omega-6 LC PUFAs include ARA, and DPAn-6.

In preferred embodiments, the beverages of the present invention comprise an antioxidant. If used, an antioxidant can be incorporated into the LC PUFA containing oil. Any antioxidant suitable for food oils preservation known in the art is compatible with the present invention, and can include vitamin E, butylhydroxytoluene (BHT), butylhydroxyanisole (BHA), tert-butylhydroquinone (TBHQ), propyl gallate (PG), vitamin C (as used herein, reference to vitamin C includes derivatives thereof), phospholipids, and natural antioxidants such as rosemary extract, and combinations thereof. Preferred antioxidants include BHA, BHT, TBHQ, a blend of BHA/BHT, and combinations thereof, and particularly, TBHQ. Amounts of antioxidant to include in the composition will vary depending on the application as determined by one skilled in the art. For example, products of the present invention comprising relatively greater amounts of omega-3 LC PUFAs (preferably having 20 or more carbons) preferably can contain higher amounts of antioxidant, such as, for example, amounts up to the maximum allowed by current United States law. Antioxidants may be added to or blended with an omega-3 LC PUFA oil by any method known in the art. Preferred amounts of antioxidant include amounts between about 0.01% and about 0.2%, and between about 0.05% and about 0.15% by weight of the oil.

The beverage stabilizing system of the invention is useful for preparing beverages having a desirable viscosity and stability. The system employs a pectin as a hydrocolloid/emulsifier which stabilizes the compositions of the invention. The pectin is preferably pre-hydrated.

In a preferred embodiment of the invention, the pectin is a natural pectin. In an alternative embodiment, the pectin is a chemically modified pectin, for example, a low methoxy pectin.

The compositions of the invention are preferably comprised of about 0.01 to about 0.5% pectin by weight of finished beverage. More preferably from about 0.02% to about 0.1%, and most preferably from about 0.05% to about 0.2% is used. More preferably, from about 0.03% to about 0.3% is used. Preferably the pectin is a finely ground powder.

Pectin is found mainly in cell walls and the intercellular spaces of vegetable tissues. Apples, plums, gooseberries and oranges are useful sources of pectin. For commercial use, pectin is extracted from shredded fruit peel or pulp by adding hot water. Pectin is a linear polymer of galacturonic acid, with a greater or lesser amount of the carboxyl groups thereof being esterified by methyl radicals. Pectins are typically classified as a function of their content in methoxy —O—CH₃ groups. Thus, pectins are distinguished as being of high methoxy (H.M.) group type (70% or more of the carboxyl groups esterified) or as pectins of low methoxy (L.M.) group type (less than 50% of the carboxyl groups are esterified) or as amidated pectins. The molecular weight of the pectins varies widely from 1,000 to 100,000 and varies as a function of the length of the chain, which may contain from several units to several hundred units of galacturonic acid. Particularly suitable for the invention are pectins of a non-gelling variety. Generally, these types of pectins do not react with calcium. Pectins that are cold-water soluble are also particularly suitable for use in the invention, since heat activation is not needed for pectins having this feature. Low methoxy pectins are also suitable for the invention, preferably in the absence of calcium. Amidated pectins are also suitable for use in the invention. Examples of suitable pectins also include, but are not limited to, TIC PRETESTED® Pectin 1694 Powder, TIC PRETESTED® Pre-Hydrated® Pectin 1694 Powder (TIC GUMS, Belcamp, Md.), CP Kelco GENU Pectin varieties (Atlanta, Ga.), and Danisco Grindsted Pectin varieties (Grinsted, Denmark).

A composition of the invention includes, in addition to the pectin and the omega-3 LC PUFA and/or omega-6 LC PUFA containing oil, a beverage component. The beverage component can comprise water, which may be carbonated or non-carbonated, a flavor, and other ingredients. In various embodiments of the invention, a flavor may be included in the oil of the invention.

The flavor can be from synthetic flavors, natural flavors, fruit flavors, botanical flavors and/or mixtures thereof. Preferably, the flavor component is a fruit flavor. The term “fruit flavor” refers to i) flavor derived from the edible reproductive part of a seed plant, especially one having a sweet pulp associated with the seed; and ii) synthetically prepared flavor made to simulate fruit flavors derived from natural sources.

The term “botanical flavor” refers to flavors derived from parts of a plant other than the fruit; i.e. derived from bean, nuts, bark, roots and leaves. Also included within the term “botanical flavor” are synthetically prepared flavors made to simulate botanical flavors derived from natural sources. Examples of such flavors include cocoa, chocolate, vanilla, coffee, kola, tea, and the like. Botanical flavors can be derived from natural sources such as essential oils and extracts, or can be synthetically prepared.

In various embodiments of the invention, the beverage component comprises a juice, including for example, juices from apple, cranberry, pear, peach, plum, apricot, nectarine, grape, cherry, currant, raspberry, gooseberry, blackberry, blueberry, strawberry, lime, lemon, orange, grapefruit, tangerine, tomato, lettuce, celery, spinach, cabbage, watercress, dandelion, rhubarb, carrot, beet, cucumber, pineapple, custard-apple, cocoa, pomegranate, guava, kiwi, mango, papaya, tamarindo, banana, watermelon, cantaloupe, and mixtures thereof can be used. Preferred juices are the citrus juices, (including orange, lemon, lime, and grapefruit) and more preferred is orange juice. Of the non-citrus juices, apple, pear, cranberry, strawberry, grape, cherry, tamarindo, pineapple, mango and kiwi are preferred. The juices and other beverages of the invention can be carbonated, if desired.

The particular amount of the flavor effective for imparting flavor characteristics to the beverages of the invention can depend upon the flavor(s) selected, the flavor impression desired, and the form of the flavor. In various embodiments of the invention, the flavor component comprises at least 0.001% by weight of the beverage to about 1%. When fruit juice is the flavor, from about 3% to about 40% is used. Up to 100% fruit juice can be used as the beverage component. For chocolate or cocoa, the amount of flavor added is from about 0.05% to about 20%. Lower levels of artificial or synthetic chocolate flavors are used than for cocoa itself.

The beverage component can comprise other additional materials including a number of materials suitable for enhancing the appearance, nutritional, organoleptic or other attributes of the beverage. Exemplary materials include colorants (natural or synthetic), vitamins, sweeteners, clouding agents and preservatives, such as benzoic acid and salts thereof sulfur dioxide, butylated hydroxyanisole, and butylated hydroxytoluene, etc. Exemplary preservatives and colors that may be used in beverages are set forth in L. F. Green, Developments in Soft Drinks Technology, Vol. 1 (Applied Science Publishers Ltd. 1978), pp. 185-186 (herein incorporated by reference). Salt, e.g. sodium chloride, and other flavor enhancers can be used to improve the flavor of the beverage. If present, such materials can constitute from about 0.01% up to about 2% of the beverage, or more as desired. Such materials can be incorporated into the exemplary beverage production described herein.

The method of the present invention includes combining an oil comprising an omega-3 LC PUFA and/or omega-6 LC PUFA with pectin and a beverage component. A flavor may be included in the oil of the invention and/or in the beverage component of the composition of the invention. The flavor may be added a various stages of the process, either before or after the emulsion is formed.

In a preferred embodiment, the method of the present invention comprises combining an omega-3 LC PUFA, an omega-6 LC PUFA, or an omega-3/omega-6 LC PUFA-containing oil with a hydrocolloid compound, such as pectin, to produce a pre-emulsion, which will be an oil in water emulsion. The pre-emulsion is then added to a beverage component to generate a beverage that is enriched with at least one omega-3 or omega-6 LC PUFA or a mixture thereof. In this manner, the omega-3 LC PUFA and/or omega-6 LC PUFA is stabilized during the storage phase of the beverage.

In an embodiment of the invention, a pre-emulsion is generated by combining pectin and water or a beverage component, preferably under high shear, and then adding the omega-3 LC PUFA and/or omega-6 containing oil with continued mixing. The entire mixture can then be subjected to homogenization, at for example, about 1000-about 10000 psi, and preferably about 2000-about 5000 psi. The pre-emulsion formed thereby can be stored and later added to a greater amount of the beverage component during beverage production.

In another embodiment of the invention, which is particularly useful in the preparation of a juice, such as a 100% juice, pectin is added to the juice, preferably under high shear, and the oil comprising at least one omega-3 LC PUFA and/or omega 6-LC PUFA is added. In this embodiment, the juice preferably has a Brix measure (measure of soluble solids) of between about 11.8 to about 13. The entire mixture (the pectin/oil/juice) is then homogenized.

In yet another embodiment of the invention, pectin is combined, under high shear, with water and an omega-3 LC PUFA containing oil is added thereto, under high shear. This mixture is then added to a desired amount of a beverage, and the mixture generated therefrom can then be subjected to homogenization. In this embodiment, the fortified/enriched beverage is subjected to homogenization.

The process of combining an oil comprising an omega-3 LC PUFA, an omega-6 LC PUFA, or a mixture thereof, with a pectin (in a liquid or solid state) to form a pre-emulsion includes combining the components and mixing to form a material that is uniform in appearance. Such mixing preferably can be done in a suitable vessel using known mixing equipment such as a Lightning mixer, a paddle mixer, a ribbon mixer or an impeller. Typically, a powder or a liquid form of pectin is added to water or to a beverage component, with mixing, until the combined material is a uniform substance. The oil is then added to the mixture. Hydrated pectin is particularly suitable for use in the methods of the invention.

Without intending to be bound by theory, the formation of the resulting LC PUFA containing oil/pectin/beverage component emulsion is believed to provide a stabilizing effect on the oil comprising omega-3 and/or omega-6 LC PUFAs, reducing the likelihood and/or the extent of oxidative degradation that the omega-3 and/or omega-6 LC PUFAs would otherwise undergo.

The field of beverage preparation is well developed and the methods of the present invention include the novel incorporation of omega-3 LC PUFA containing oil-pectin mixture described herein into beverages, at various stages of beverage production. A description of beverage preparation technology, including formulations and product preparation processing is, for example, contained in L. F. Green, Developments in Soft Drinks Technology, Vol. 1, (Applied Science Publishers Ltd. 1978), pp. 102-107 (herein incorporated by reference).

The beverages of the present invention can be prepared by standard beverage formulation techniques. Beverage making techniques, when appropriately modified, are also applicable to carbonated beverages. Diet beverages containing noncaloric and artificial sweeteners can also be prepared by appropriate modification. The terms “beverage” and “beverage component” are intended to include drinkable liquids, other than milk. Beverages can include dry beverage mixes which are made by mixing flavors, sweeteners, and emulsifier system and any optional ingredients. The ingredients are typically added to water and mixed in conventional equipment. One skilled in the art can ascertain the mixing conditions required to prepare a beverage of the appropriate viscosity. Generally, the higher shear mixer used, the more viscous the beverage will be.

In an exemplary method, a combination of pectin and an omega-3 LC PUFA containing oil is added to a beverage component, such as a beverage concentrate or a beverage syrup. A beverage syrup is generally produced by adding sugar and water to a beverage concentrate. The beverage syrup is then mixed with an appropriate quantity of water to form the finished beverage. In various embodiments, the weight ratio of water:syrup is typically from about 3:1 to about 8:1.

To make a carbonated beverage carbon dioxide can be introduced either into the water mixed with the beverage syrup or into the drinkable diluted beverage to achieve carbonation. The beverage can be sealed in a container such as a bottle or can by techniques known in the art.

The amount of carbon dioxide in the beverage depends upon the particular flavor system used and the amount of carbonation desired. Usually, carbonated beverages of the present invention contain from 1.0 to 4.5 volumes of carbon dioxide. Preferred carbonated beverages contain from 2 to 3.5 volumes of carbon dioxide. One volume of carbon dioxide is the amount of carbon dioxide absorbed by any given quantity of water at 60 degrees F., atmospheric pressure. A volume of gas occupies the same space, as does the water by which it is absorbed.

Acids such as citric, malic, ascorbic, tartaric or phosphoric acid, that can form a part of the flavor can be added at various points in these process. Usually, the acids are added with the fruit juice or other flavors.

The beverages of the present invention preferably can be conventionally packaged. In preferred embodiments, the products of the present invention are stored under appropriate conditions to reduce oxidative degradation. Many methods to effectuate such storage conditions are known in the art and are suitable for use with the present invention. A preferred method by which to reduce or minimize oxidative degradation is to package the beverages aseptically prior to storage.

In an aseptic packaging process, a liquid food or beverage is typically sterilized outside the package using an ultra-high temperature (UHT) process that rapidly heats, and then cools the product before filling. The processing equipment allows the time (generally 3 to 15 seconds) and temperature (195° to 285° F.) to be tailored to place the appropriate amount of thermal stress on the product.

Suitable aseptic packaging includes Tetra Brik® from Tetra Pak®, which is particularly useful for juices. Aseptic packaging typically keeps beverages fresh for months, and eliminates the need for refrigeration.

The present invention, while disclosed in terms of specific methods, products, and organisms, is intended to include all such methods, products, and organisms obtainable and useful according to the teachings disclosed herein, including all such substitutions, modifications, and optimizations as would be available to those of ordinary skill in the art. The following examples and test results are provided for the purposes of illustration and are not intended to limit the scope of the invention.

### EXAMPLE 1

This example shows orange juice prepared in accordance with the present invention.

Orange juice fortified with DHA was produced as described herein. Four variations, including a control, were produced evaluating two separate emulsifiers, pectin and xanthan gum.

The variety of hydrocolloid/emulsifier in the first three variations set forth in Table 1 is Pre-Hydrated® pectin 1694 supplied by TIC gums (Belcamp, Md.). The pectin was pre-hydrated, so that it would not clump when added to aqueous solutions.

Xanthan gum (CP Kelco T22) was used in the formulation 4, as set forth in Table 1. Ascorbic acid (at 0.15% of the total formulation) was added to improve taste and act as an antioxidant.

The orange juice was monitored for 20 weeks under refrigeration. The packaging was completely aseptic, and the orange juice packaging size was 250 ml.

Variations 2 and 3 differed in that 3 contained the chelating agent EDTA. No significant difference was found in sensory panels and peroxide values for the two samples, suggesting no advantage to formulating with EDTA in this example.

TABLE 1

| | |
|---|---|
| Variation | Ingredient % |

| | | | |
|---|---|---|---|
| 1) | Orange Juice | 99.55 | |
| | Pectin | 0.3 | |
| | Ascorbic Acid | 0.15 | |
| 2) | Orange Juice | 99.494 | |
| | Pectin | 0.3 | |
| | Martek DHA ™-S | 0.056 | 50 mg |
| | (Martek Biosciences | | DHA/serving |
| | Corporation, Columbia, | | |
| | MD) | | |
| | Ascorbic Acid | 0.15 | |
| 3) | Orange Juice | 99.494 | |
| | Pectin | 0.3 | |
| | Martek DHA ™-S | 0.056 | 50 mg |
| | (Martek Biosciences | | DHA/serving |
| | Corporation, Columbia, | | |
| | MD) | | |
| | Ascorbic Acid | 0.15 | |
| | EDTA | 28 ppm | |
| 4) | Orange Juice | 99.714 | |
| | Xanthan Gum | 0.08 | |
| | Martek DHA ™-S | 0.056 | 50 mg |
| | (Martek Biosciences | | DHA/serving |
| | Corporation, Columbia, | | |
| | MD) | | |
| | Ascorbic Acid | 0.15 | |
| | EDTA | 28 ppm | |

The production process began by weighing 35 lbs of water into a 70 lb size vessel. The pre-hydrated pectin was then added to the water and mixed under high shear using a Lightning mixer at 3000-10000 rpm, for about 2 minutes or until the solution was smooth and homogeneous. Martek DHA™-S, a DHA-containing algal oil was then added to the pectin/water mixture and further mixed under high shear for one minute. In a separate balance tank frozen orange concentrate was thawed and then the thawed orange juice concentrate was weighed and placed under light agitation for about 45 minutes.

The water/algal oil/pectin solution in the vessel was then added to the orange juice concentrate in the balance tank while slowly mixing. Water was then added to the balance tank until the solution was diluted to 11.8-12 Brix. Once the desirable Brix strength was attained, the agitation speed was increased and the ascorbic acid and EDTA were added to the balance tank and mixed under light agitation (approximately 200 rpm) for one to two minutes. The orange juice was then pumped from the balance tank via progressive cavity pump to the first plate heat exchanger. The temperature of the orange juice was raised to 170 F and it flowed to the steam injector where it was held at 197 F for 3.5 seconds. The orange juice was then passed through a vacuum chamber to the homogenizer via centrifugal pump while at 170 F. The homogenizer placed 1500 psi of pressure on the product during the first stage of homogenization and 500 psi on the product during the second stage of homogenization. The orange juice was then subjected to a second plate heat exchanger where the temperature dropped to 40 F. The orange juice was then collected in a holding tank and packaged in aseptic 250 ml tetrabrix containers. The orange juice containers were collected and stored under refrigeration, at approximately 40 F.

### EXAMPLE 2

This example demonstrates the stability of fortified orange juice prepared in accordance with Example 1.

DHA recovery was determined using a modification of AOAC method 996.06. The orange juice samples were freeze dried. Fatty acids were transesterified in situ with 1.5 N HCl in methanol in the presence of toluene and an internal standard (methyl nonadecanoate, C19:0). The resultant fatty acid methyl esters (FAME's) were extracted with toluene. The FAME's were separated, identified, and quantitated by gas-liquid chromatography (GLC).

Duplicate analyses were performed for each sample set forth in Table 1. A statistical process control sample was analyzed with the orange juice and was found to be within the expected range. The DHA check standard was also within the expected range. A three point internal standard (C19:0) calibration was used to quantitate DHA. The results are set forth in Table 2.

TABLE 2

| Control | Average recovery | recovery % |
|---|---|---|
| OJ 2 | 40.47 | 86.84 |
| OJ 3 | 42.7 | 91.2 |
| OJ 4 | 43.97 | 93.74 |
| Average | 42.38 | 90.59 |

### EXAMPLE 3

This example evaluates the production of peroxide values in orange juice prepared in accordance with the invention.

The samples of the fortified orange juice, prepared in accordance with Example 1, were tested for peroxides at a frequency of once every month for four months. The peroxide and alkenal values are early indicators of oxidation. To measure the peroxide value a PeroxySafe™ assay was used. (Safetest® Inc., Temp, Ariz.). The assay is based on hydroperoxide-meditated oxidation of acidified iron, which gives off a color and the degree of that color is then measured and indicates the amount of oxidation.

The peroxide results after up to 4 months of storage under refrigeration were below detection limits of the method (approximately 0.15 meq/kg or 0.15 milliequivalents/kilogram).

### EXAMPLE 4

This example shows the sensory evaluation of orange juice product prepared in accordance with the present invention.

Sensory Panel 1

Sensory evaluations of orange juice prepared in accordance with Example 1 (listed in Table 3) were conducted using the difference-from-control test. Overall flavor difference was measured on a 0-10 point descriptive scale where 0=no difference and 10=very large difference from control sample. Participants described differences perceived, if any. Results are set forth in Tables 4 (refrigerated orange juice) and Table 5 (orange juice stored in aseptic packaging and at room temperature).

TABLE 3

| Variable | mg/250 ml | | | |
|---|---|---|---|---|
| Name | box | emulsifier | EDTA | |
| Control | 0 | pectin | no | DHA successfully |
| OJ 2 | 50 | pectin | no | incorporated |
| OJ 3 | 50 | pectin | yes | into these variables |
| OJ 4 | 50 | xanthan | yes | over allotted |
| | | | | shelf life study. |

TABLE 4

| | | | | | Discussion/ |
|---|---|---|---|---|---|
| Weeks | | OJ 2 | OJ 3 | OJ 4 | Observations |
| 12 days | N | 19 | 19 | 19 | no significant differences |
| | Average | 0.7 | 1.1 | 1.8 | from control sample |
| | sd | 1.0 | 1.6 | 2.0 | |
| 3 | N | 19 | 19 | 19 | no significant differences |
| | Average | 0.5 | 1.3 | 1.4 | from control sample |
| | sd | 0.7 | 1.3 | 1.3 | |
| 7 | N | 23 | 23 | 23 | no significant differences |
| | Average | 0.5 | 0.6 | 2.0 | from control sample |
| | sd | 0.7 | 1.0 | 2.4 | |
| 10 | N | 19 | 19 | 19 | no significant differences from |
| | Average | 1.1 | 0.9 | 0.7 | control sample - no differences |
| | sd | 1.5 | 1.3 | 1.0 | attributed to fishy off |

TABLE 5
(Room Temperature)

| | | | | | Discussion/ |
|---|---|---|---|---|---|
| Week | | OJ 2 | OJ 3 | OJ 4 | Observations |
| 5 | N | 15 | 15 | 15 | no significant differences |
| | Average | 0.9 | 0.4 | 0.7 | from control sample |
| | sd | 1.0 | 1.1 | 1.1 | |
| 9 | N | 18 | 18 | 18 | no significant differences |
| | Average | 1.3 | 1.3 | 2.1 | from control sample |
| | sd | 1.3 | 1.3 | 2.1 | |

No significant differences from the control were detected in any of the samples tested. The refrigerated samples were stable up to ten and a half weeks and the room temperature samples were stable up to nine weeks.

Sensory Panel 2

Additional sensory panels were conducted for twenty weeks at five separate time intervals, at 1.5, 7, 12, 16.5, and 20 weeks of storage of the juice under refrigeration. The results are set forth in Table 6. The tests were all compare to control format in which the panelist first tasted the control and then tasted the treatment variation to detect differences. The fourth variation, which had xanthan gum as the hydrocolloid, was pulled from the panel at 16.5 weeks due to loss of solids suspension. The variables that used pectin as the hydrocolloid did extremely well, with none of the panelists being able to detect a fishy/marine flavor in any panels. In many of the panels the panelists preferred the taste of the treatment variation to the control.

TABLE 6
Progression of sensory scores

| Variable | # 2 | St Dev | # 3 | St Dev | # 4 | St Dev | Weeks |
|---|---|---|---|---|---|---|---|
| T0 | 0.625 | 0.806 | 0.438 | 0.727 | 0.625 | 1.15 | 1.5 |
| T1 | 1.08 | 1.56 | 0.67 | 1.44 | 0.583 | 0.996 | 7 |
| T2 | 0.571 | 0.85 | 0.714 | 0.91 | 1.43 | 1.79 | 12 |
| T3 | 1.09 | 1.7 | 0.273 | 0.65 | 0.182 | 0.57 | 16.5 |
| T4 | 0.33 | 0.65 | 0.58 | 0.996 | | | 20 |

All technical references, patent applications and patents cited herein are hereby incorporated herein by reference in their entirety, as if fully set forth herein.

The principles, preferred embodiments and modes of operation of the present invention have been described in the foregoing specification. The invention which is intended to be protected herein should not, however, be construed as limited to the particular forms disclosed, as these are to be regarded as illustrative rather than restrictive. Variations and changes may be made by those skilled in the art without departing from the spirit of the present invention. Accordingly, the foregoing best mode of carrying out the invention should be considered exemplary in nature and not as limiting to the scope and spirit of the invention as set forth in the appended claims.

1. A composition comprising:
an oil comprising at least one long chain polyunsaturated fatty acid;
pectin; and
a beverage component,
wherein said composition is free of alginate and of calcium gluconate.

2. The composition claim 1, wherein the oil is from a microbial source.

3. The composition of claim 2, wherein the microbial source is a microorganism selected from the group consisting of algae, bacteria, fungi, and protists.

4. The composition of claim 3, wherein the microorganism is an algae.

5. The composition of claim 3, wherein the microbial source is an oleaginous microorganism.

6. The composition of claim 5, wherein the microorganism is selected from the group consisting of the genus Thraustochytrium, microorganisms of the genus Schizochytrium, microorganisms of the genus Althomia, microorganisms of the genus Aplanochytrium, microorganisms of the genus Japonochytrium, microorganisms of the genus Labyrinthula, microorganisms of the genus Labyrinthuloides, microorganisms of the genus Crypthecodinium, microorganisms of the genus Mortierella, and mixtures thereof.

7. The composition of claim 6, wherein the microorganism is selected from the group consisting of microorganisms of the genus Thraustochytrium, microorganisms of the genus Schizochytrium, microorganisms of the genus Crypthecodinium, microorganisms of the genus Mortierella and mixtures thereof.

8. The composition of claim 1, wherein oil is from a plant source.

9-10. (canceled)

11. The composition of claim 1, wherein the oil is from an animal source.

12. (canceled)

13. The composition of claim 1, wherein the at least one long chain polyunsaturated fatty acid is selected from the group consisting of an omega-3 LC PUFA, an omega-6 LC PUFA, and a mixture thereof.

14. The composition of claim 13, wherein the omega-3 LC-PUFA or omega-6 LC PUFA is selected from the group consisting of docosahexaenoic acid, eicosapentaenoic acid, docosapentaenoic acid, arachidonic acid and mixtures thereof.

15. The composition of claim 1, wherein the oil comprises at least about 20% omega-3 LC PUFAs.

16. The composition of claim 1, wherein the oil comprises at least about 30% omega-3 LC PUFAs.

17-21. (canceled)

22. The composition of claim 1, wherein the oil comprises between about 5 mg and about 1000 mg of omega-3 LC PUFA or omega-6 LC PUFA per serving.

23. The composition of claim 1, wherein the oil comprises between about 5 mg and about 250 mg of omega-3 LC PUFA or omega-6 LC PUFA per serving.

24-26. (canceled)

27. The composition of claim 1, wherein the oil is encapsulated in a powder.

28. The composition of claim 1, wherein the pectin is selected from the group consisting of a natural pectin and a modified pectin.

29. The composition of claim 1, further comprising an antioxidant.

30. The composition of claim 29, wherein the antioxidant is selected from vitamin E, butylhydroxytoluene (BHT), butylhydroxyanisole (BHA), tert-butylhydroquinone (TBHQ), propyl gallate (PG), vitamin C, phospholipids, natural antioxidants, and combinations thereof.

31. The composition of claim 29, wherein the antioxidant is present in an amount between about 0.01 and about 0.2 by weight of the oil.

32. The composition of claim 1, wherein the pectin is present in an amount of about 0.020% to 0.10% by weight of the composition.

33-35. (canceled)

36. The composition of claim 1, wherein the beverage component is selected from the group consisting of a natural flavor and an artificial flavor.

37. The composition of claim 1, wherein said beverage component is selected from the group consisting of fruit juice, fruit flavor, fruit concentrate, tea, carbonated water, protein and mixtures thereof.

38. The composition of claim 37, wherein the beverage component is fruit juice.

39. The composition of claim 38, wherein the fruit juice is orange juice.

40. The composition of claim 38, wherein the fruit juice is concentrated.

41. A composition comprising:
an oil comprising at least one LC PUFA;
pectin; and
water or an aqueous phase,
wherein said composition is free of alginate and of calcium gluconate.

42-75. (canceled)

76. A process for preparing the composition of claim 1, comprising:
(a) combining an oil comprising at least one long chain polyunsaturated fatty acid and pectin;
(b) adding the mixture produced in (a) to the beverage component.

77-84. (canceled)

85. A composition produced by the process of claim 76.

86. A process for preparing the composition of claim 1, comprising:
(a) combining an oil comprising a long chain fatty acid selected from the group consisting of an omega-3 LC PUFA, an omega-6 LC PUFA, and a mixture thereof and pectin;
(b) adding the mixture produced in (a) to the beverage component;
(c) subjecting the mixture produced in (b) to homogenization.

87-92. (canceled)
